(19) United States
(12) Patent Application Publication
(10) Pub. No.: US 2026/0248450 A1
(43) Pub. Date: Aug. 27, 2026

(54) SYSTEM

(71) Applicant: SoftBank Group Corp., Tokyo (JP)

(72) Inventor: Takato YAMAZAKI, Tokyo (JP)

(73) Assignee: SoftBank Group Corp., Tokyo (JP)

(21) Appl. No.: 19/539,101

(22) Filed: Feb. 13, 2026

(30) Foreign Application Priority Data
Feb. 21, 2025 (JP) 2025-026966

(51) Int. Cl. A61B 5/00; A61B 5/16; G06F 40/30

(52) U.S. Cl. CPC A61B 5/486; A61B 5/165; A61B 5/7264; G06F 40/30

(57) ABSTRACT

A system according to an embodiment includes an acquisition unit, an analysis unit, a questioning unit, and an advice unit. The acquisition unit acquires health data. The analysis unit analyzes the data acquired by the acquisition unit and detects an abnormal numerical value or pattern. The questioning unit asks a user a question based on the abnormal numerical value or pattern detected by the analysis unit. The advice unit identifies a cause based on an answer of the user obtained by the questioning unit and provides advice.

### CROSS-REFERENCE TO RELATED APPLICATION

The present application claims priority to and incorporates by reference the entire contents of Japanese Patent Application No. 2025-026966 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, a cause or meaning of a numerical value of health data acquired by a smart watch is not explained to a user, and there is room for improvement.

### SUMMARY OF THE INVENTION

A system according to an embodiment includes an acquisition unit, an analysis unit, a questioning unit, and an advice unit. The acquisition unit acquires health data. The analysis unit analyzes the data acquired by the acquisition unit and detects an abnormal numerical value or pattern. The questioning unit asks a user a question based on the abnormal numerical value or pattern detected by the analysis unit. The advice unit identifies a cause based on an answer of the user obtained by the questioning unit and provides advice.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5 th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### FIRST EMBODIMENT

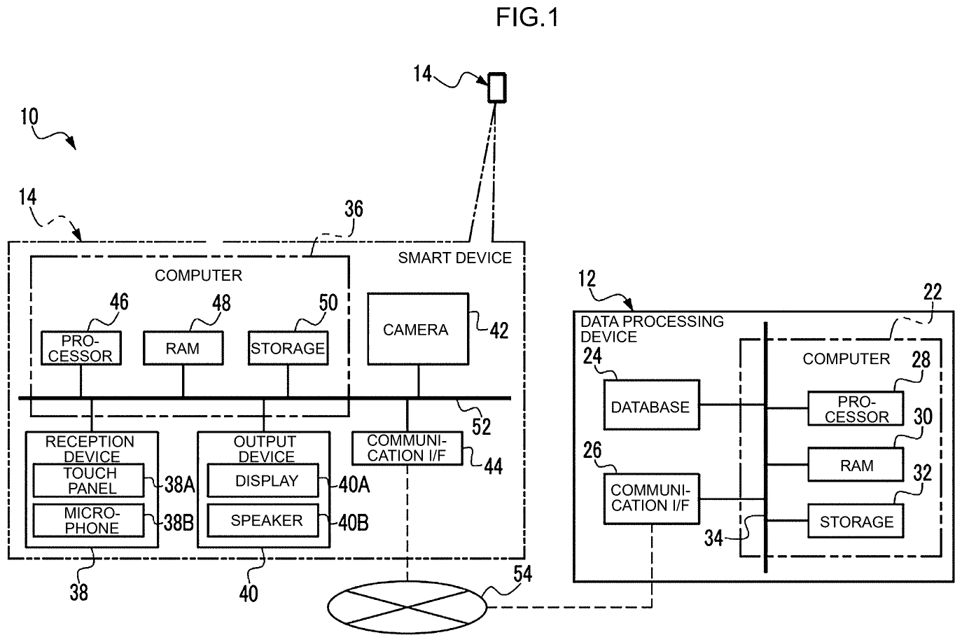

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

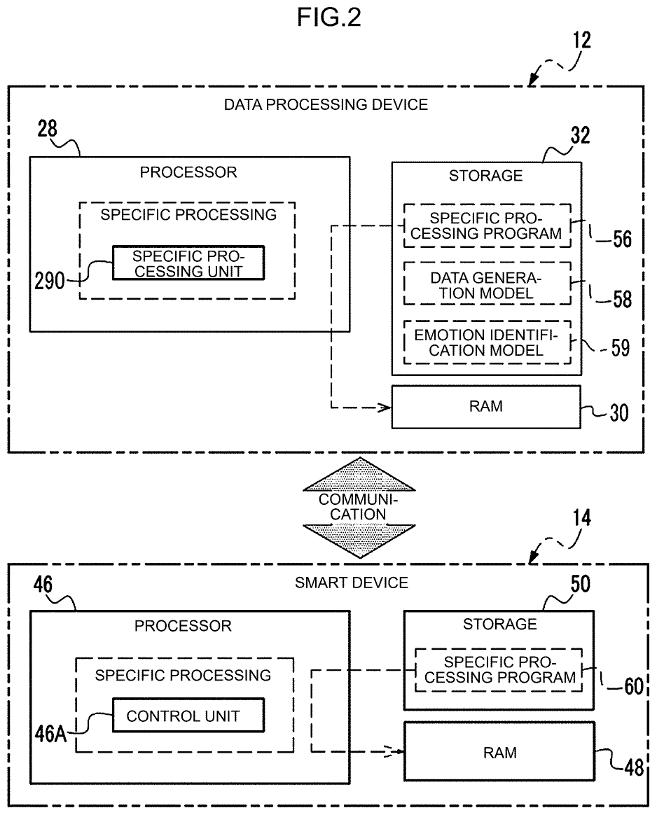

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### EXAMPLE OF THE EMBODIMENT

A health assistant system according to an embodiment of the present invention is a system that provides health advice based on sensor values of a smartwatch. This system acquires health data, analyzes it, asks a user a question, and provides appropriate advice. For example, when a heart rate rises rapidly, the system analyzes data of that time zone in detail and asks the user what event occurred at that time. Based on an answer from the user, the system identifies a cause of a spike in heart rate and provides appropriate advice. For example, if the user was exercising, the system determines that the rise in heart rate is due to exercise and conveys that there is no particular problem. On the other hand, if the user was feeling stress, the system provides advice on stress management. For example, the system proposes a breathing method or a stretching method for relaxing. Also, if the user was eating, the system advises an improvement point regarding a meal since meal content or meal timing may have affected the heart rate. In this way, the system supports the user's health management by analyzing the user's health state in detail based on the sensor values of the smartwatch and providing appropriate advice. For example, when a spike in heart rate is observed periodically, the system can recommend a review of lifestyle habits or a medical examination by a doctor. In addition, by understanding their own health state more deeply, the user's health awareness increases, and they can lead a healthier life. Thereby, the health assistant system can analyze the user's health state in detail and provide appropriate advice. Specifically, this system includes a data processing pipeline that continuously acquires biological signals obtained from a photoplethysmography (PPG) sensor or a 3-axis acceleration sensor built into a wearable device as time-series data. The system performs noise removal filtering and normalization processing on the acquired time-series data (for example, pulse wave waveform data and acceleration vector data acquired at a sampling rate of 50 Hz), and then inputs the data to a time-series analysis model such as a Recurrent Neural Network (RNN) or Long Short-Term Memory (LSTM). This AI model outputs an anomaly score (a probability value in a range of 0.0 to 1.0) indicating a degree of deviation from a normal biological rhythm based on the input multidimensional time-series data (for example, a tensor of [batch size, number of time steps, number of feature dimensions]). When the anomaly score exceeds a predetermined threshold (for example, 0.8), the system generates a trigger signal and activates a dialogue engine using a Large Language Model (LLM). The dialogue engine receives a context of the detected abnormality (time, duration, rate of change in intensity) as input and generates a natural language question sentence for confirming a situation with the user (for example, “Your heart rate rose around 2:00 PM, were you doing any special activity?”). An answer text from the user is analyzed by a natural language processing module and classified into an event category (exercise, stress, meal, etc.). By integrating this classification result and an analysis result of the sensor data, the system executes inference processing to identify a physiological or psychological cause of the spike in heart rate. For example, if the heart rate rises even though the acceleration data indicates a stationary state and the user answer is “in a meeting”, the system determines it as “mental stress” and generates breathing method guidance data for making the parasympathetic nerve dominant and transmits it to a user terminal. In this way, the system not only monitors numerical values but also elucidates a causal relationship for each individual event by integrally analyzing sensor data and user context with a sophisticated AI model, thereby achieving a technical effect of presenting specific and actionable health improvement actions.

The health assistant system according to the embodiment comprises an acquisition unit, an analysis unit, a questioning unit, and an advice unit. The acquisition unit acquires health data. The health data includes, for example, a heart rate, blood pressure, body temperature, and the like, but is not limited to such examples. The acquisition unit acquires the health data using, for example, a sensor of a smartwatch. Also, the acquisition unit can acquire data from a smartphone of the user or other wearable devices. The analysis unit analyzes the data acquired by the acquisition unit and detects an abnormal numerical value or pattern. The analysis unit analyzes, for example, heart rate data and detects an abnormal pattern. The abnormal pattern includes, for example, a rapid fluctuation in heart rate or a sustained high heart rate, but is not limited to such examples. The analysis unit can analyze data using AI to detect an abnormal numerical value or pattern. The questioning unit asks the user a question based on the abnormal numerical value or pattern detected by the analysis unit. The questioning unit asks the user, for example, what event occurred at that time. A format of the question includes, for example, a selection type or a free description type, but is not limited to such examples. The questioning unit can ask the user a question using AI. The advice unit identifies a cause based on an answer of the user obtained by the questioning unit and provides appropriate advice. The advice unit proposes, for example, a breathing method or a stretching method for relaxing. Also, the advice unit can propose an improvement point regarding meal content or meal timing. The advice unit can provide appropriate advice to the user using AI. Thereby, the health assistant system according to the embodiment can analyze the user's health state in detail and provide appropriate advice. Specifically, the acquisition unit has a sensor fusion interface that connects to a plurality of heterogeneous sensor devices (smartwatch, smart ring, environmental sensor, etc.) via a wireless communication protocol such as Bluetooth Low Energy (BLE) and integrates packet data transmitted asynchronously. The acquisition unit performs missing value imputation and timestamp synchronization processing on received raw data (Raw Data), and converts it into a data frame of a unified format processable by the analysis unit. The analysis unit implements a deep learning-based anomaly detection model (for example, an autoencoder or Variational Autoencoder: VAE), and calculates a reconstruction error of input data using health data during a normal time as training data. The input data is, for example, a multivariate time-series vector including a heart rate, skin temperature, and electrodermal activity (EDA) for the past 24 hours, and output data is reconstruction data having the same dimensions as the input data and a scalar value of the reconstruction error. When the reconstruction error shows a statistically significant deviation, the analysis unit sets an abnormality flag and passes the feature vector to the questioning unit. Based on the received feature vector and a type of abnormality (e.g., sudden tachycardia), the questioning unit generates a context-dependent question considering a current situation of the user and a past dialogue history using Generative AI. The advice unit includes an inference engine that receives multimodal input (text+sensor numerical value) integrating the user's answer and the analysis result, and determines an optimal intervention measure (advice) by referring to a Knowledge Graph. With this configuration, the system identifies complex health risk factors that are difficult to detect with conventional rule-based systems, and realizes precise health management support optimized for the physiological characteristics of the individual user.

The advice unit can propose a breathing method or a stretching method for relaxing. The breathing method for relaxing includes, for example, deep breathing, abdominal breathing, and the like, but is not limited to such examples. The advice unit proposes, for example, a method of deep breathing. Deep breathing is performed by inhaling slowly and deeply and exhaling slowly. Also, the advice unit can propose a method of abdominal breathing. Abdominal breathing is performed by inhaling so as to inflate the abdomen and exhaling so as to retract the abdomen. The stretching method includes, for example, yoga poses, muscle stretching methods, and the like, but is not limited to such examples. The advice unit proposes, for example, a yoga pose. The yoga pose includes, for example, a cat pose, a child's pose, and the like. The cat pose is performed by getting on all fours and rounding or arching the back. The child's pose is performed by sitting with knees bent, bending forward, and touching the forehead to the floor. Thereby, by proposing a breathing method or a stretching method for relaxing, stress management of the user can be supported. Part or all of the above-described processing in the advice unit may be performed using AI or may be performed without using AI. Specifically, the advice unit analyzes a current biological signal (Heart Rate Variability: HRV) of the user in real time and forms a biofeedback loop that estimates an autonomic nerve balance (ratio of sympathetic nerve to parasympathetic nerve) of the user. The advice unit generates an optimal breathing rhythm (for example, a pattern of 4 seconds inhalation, 7 seconds hold, 8 seconds exhalation) according to an estimated stress level, and transmits it to the user's wrist as a vibration pattern using a haptic (tactile) feedback function of the smartwatch. Furthermore, in the proposal of stretching, the advice unit operates a pose estimation AI model (for example, a model based on PoseNet or MediaPipe) that estimates a current posture of the user using input data from a camera of the smartphone or a motion sensor (accelerometer/gyroscope) of the smartwatch. This AI model takes an image frame or time-series data of the motion sensor as input and outputs coordinate data of major joint points (key points) of the body. The advice unit calculates a difference (Euclidean distance or angular difference) between the output joint point coordinates and a template of an ideal yoga pose, and feeds back a specific correction instruction such as “Please round your back a little more” to the user in real time by voice or screen display. In this way, the system not only presents information but also guides the user's body movement by closed-loop control using sensors, and dynamically adjusts advice while quantitatively confirming improvement in physiological indices (HRV, etc.), thereby realizing a reliable stress reduction effect.

The advice unit can propose an improvement point regarding meal content or meal timing. The improvement point regarding meal content includes, for example, nutritional balance, calorie intake, and the like, but is not limited to such examples. The advice unit proposes, for example, a nutritionally balanced meal. The nutritionally balanced meal includes, for example, vegetables, fruits, proteins, carbohydrates, and the like. Also, the advice unit can propose management of calorie intake. The management of calorie intake includes, for example, calculating daily calorie intake and keeping it within an appropriate range. The improvement point regarding meal timing includes, for example, meal intervals, meal time zones, and the like, but is not limited to such examples. The advice unit proposes, for example, keeping meal intervals appropriate. By keeping meal intervals appropriate, rapid fluctuations in blood glucose levels can be prevented. Also, the advice unit can propose adjusting meal time zones. For example, it proposes avoiding eating late at night and having an early dinner. Thereby, by proposing an improvement point regarding meal content or meal timing, the user's health management can be supported. Part or all of the above-described processing in the advice unit may be performed using AI or may be performed without using AI. Specifically, the advice unit includes an image recognition AI model (for example, Convolutional Neural Network: CNN) that takes image data of a meal taken by the user as input. This CNN model extracts feature quantities from an input RGB image tensor (for example, [224, 224, 3]), segments a food region in the image, and outputs a food category (e.g., steak, salad, rice) of each region and its estimated amount (in grams). Furthermore, the advice unit collates the estimated food information with a nutrition database to calculate total calories, macronutrients (PFC balance), vitamins, and mineral content. Also, the advice unit simulates an influence of current meal content and timing on a postprandial blood glucose level using a time-series prediction model (for example, LSTM or Transformer) that has learned a correlation between the user's past blood glucose level data (obtained from CGM: Continuous Glucose Monitoring, etc.) and meal timing. This prediction model takes a meal content vector and intake time as input and outputs a blood glucose level transition prediction curve for two hours after the meal. When a predicted blood glucose spike exceeds a threshold, the advice unit generates a specific behavioral change nudge such as “Eat vegetables first (Veggie First)” or “Take a 15-minute walk after the meal”. Thereby, the system realizes Precision Nutrition based on individual metabolic characteristics, going beyond general nutritional guidance.

The analysis unit can analyze heart rate data and detect an abnormal pattern. The heart rate data includes, for example, a resting heart rate, an exercise heart rate, and the like, but is not limited to such examples. The analysis unit analyzes, for example, data of the resting heart rate and detects an abnormal pattern. The abnormal pattern includes, for example, a rapid fluctuation in heart rate, a sustained high heart rate, and the like. The analysis unit can analyze the heart rate data using AI to detect an abnormal pattern. For example, the analysis unit performs analysis using an AI model that takes the heart rate data as input and outputs an abnormal pattern. Thereby, by analyzing the heart rate data and detecting an abnormal pattern, the user's health state can be grasped. Specifically, the analysis unit takes time-series data of heartbeat intervals (R-R intervals) as input and executes a dedicated algorithm for performing Heart Rate Variability (HRV) analysis. The analysis unit calculates time domain indices (SDNN, RMSSD) and frequency domain indices (LF/HF ratio) to quantify a state of autonomic nerve function. Furthermore, the analysis unit uses an anomaly detection model trained by unsupervised learning (for example, One-Class SVM or Isolation Forest, or LSTM autoencoder). Input to this model is a heart rate sequence cut out by a sliding window method (for example, window size 60 seconds, overlap 50%), and standardization is performed as preprocessing. The model outputs an anomaly score for each time step based on a distribution density in a feature space of the input sequence or a reconstruction error. When the anomaly score exceeds a dynamically set threshold, the analysis unit assigns an abnormality label such as “suspicion of atrial fibrillation” or “acute stress reaction” and transmits an alert signal to a subsequent processing module. By this processing, it becomes possible to detect minute disturbances in heart rhythm that humans do not notice or deviations from long-term trends at an early stage, and to capture signs of serious health risks.

The questioning unit can ask the user what event occurred at that time. The event includes, for example, exercise, a meal, a stressful occurrence, and the like, but is not limited to such examples. The questioning unit asks the user, for example, whether they were exercising at that time. If they were exercising, it can be determined as a rise in heart rate due to exercise. Also, the questioning unit can ask the user whether they were eating at that time. If they were eating, it can be determined that meal content or meal timing may have affected the heart rate. Furthermore, the questioning unit can ask the user whether they were feeling stress at that time. If they were feeling stress, advice on stress management can be provided. Thereby, by asking the user what event occurred at that time, the cause of the abnormal numerical value can be identified. Part or all of the above-described processing in the questioning unit may be performed using AI or may be performed without using AI. Specifically, the questioning unit starts a dialogue generation process using a Large Language Model (LLM) triggered by a timestamp and a type (e.g., rapid heart rate rise) of an abnormal event notified from the analysis unit. The questioning unit inputs an instruction (prompt) such as “Role: Health Assistant”, “Context: Heart rate rose to 120 bpm at 3:00 PM”, and “Constraint: Short sentence not burdening the user” to the LLM using prompt engineering technology. Based on this input, the LLM generates and outputs a natural and empathetic question sentence (e.g., “You seem to have had a little palpitation, did you move in a hurry?”). Furthermore, the questioning unit receives an answer (voice or text) from the user and performs intent extraction (Intent Classification) and slot filling (Slot Filling) using a Natural Language Understanding (NLU) module. For example, when the user answers “I was arguing with my boss”, the NLU module outputs structured data of “Event Type: Social Stress” and “Intensity: High”. This structured data is input to a subsequent inference engine and used to definitively diagnose that the cause of the heart rate rise is not “physical load” but “psychological load”. In this way, the dialogue processing using AI complements context information that cannot be determined only by sensor data, reduces false positives, and dramatically improves the accuracy of advice.

The advice unit can recommend a review of lifestyle habits or a medical examination by a doctor when a spike in heart rate is observed periodically. The review of lifestyle habits includes, for example, sleep habits, exercise habits, dietary habits, and the like, but is not limited to such examples. The advice unit proposes, for example, a review of sleep habits. The review of sleep habits includes, for example, securing regular sleep time and maintaining a comfortable sleep environment. Also, the advice unit can propose a review of exercise habits. The review of exercise habits includes, for example, continuation of moderate exercise and review of types of exercise. Furthermore, the advice unit can propose a review of dietary habits. The review of dietary habits includes, for example, a balanced diet and appropriate meal timing. Thereby, when a spike in heart rate is observed periodically, the user's health management can be supported by recommending a review of lifestyle habits or a medical examination by a doctor. Part or all of the above-described processing in the advice unit may be performed using AI or may be performed without using AI. Specifically, the advice unit includes a trend analysis engine that analyzes a log of health data accumulated over a long period (for example, one month or more). This engine classifies occurrence patterns of heart rate spikes using a time-series clustering algorithm (for example, k-means method or DBSCAN). For example, if spikes are concentrated on a specific day of the week or time zone (e.g., Monday morning), the advice unit performs inference suspecting “social jet lag” or “start-of-week stress”. Furthermore, the advice unit uses a prediction model (for example, Random Forest or Gradient Boosting Decision Tree: GBDT) that predicts a health risk of the user, and calculates a future health risk score (e.g., cardiovascular disease risk probability) if the current lifestyle habits are continued. When this risk score exceeds a predetermined threshold, the advice unit has a function of generating a message recommending a visit to a medical institution and automatically generating a summary report (PDF etc. including a heart rate variability graph, abnormality occurrence frequency, and a list of related events) to be shown to a doctor. Thereby, a bridge from daily health management to professional medical intervention is smoothly performed, contributing to the extension of the user's healthy life expectancy from the viewpoint of preventive medicine.

The acquisition unit can estimate an emotion of the user and adjust an acquisition timing of the health data based on the estimated emotion of the user. For example, when the user is feeling stress, the acquisition unit can increase an acquisition frequency of the health data until the stress is alleviated. Also, when the user is relaxed, the acquisition unit can decrease the acquisition frequency of the health data to reduce the burden on the user. Furthermore, when the user is exercising, the acquisition unit can acquire the health data at a peak of the exercise to collect detailed data. Thereby, by adjusting the acquisition timing of the health data based on the emotion of the user, more appropriate data can be acquired. The estimation of emotion is realized using an emotion estimation function using, for example, an emotion engine or Generative AI. The Generative AI is text generation AI (for example, LLM), multimodal Generative AI, or the like, but is not limited to such examples. Part or all of the above-described processing in the acquisition unit may be performed using AI or may be performed without using AI. Specifically, the acquisition unit executes an emotion recognition AI model (for example, a Transformer-based voice/language integrated model) that takes the user's voice data (tone, pitch, speed) and text input (chat log) as multimodal input. This model outputs a vector representing an emotional state (e.g., a 5-dimensional probability distribution of [joy, anger, sadness, relaxation, stress]) from the input data. When a probability value of “stress” is high in the output emotion vector, the acquisition unit issues a sampling rate change command to a sensor control module. The acquisition unit switches from heart rate measurement once a minute during normal times to a continuous measurement mode of once a second during stress detection, and collects detailed data of Heart Rate Variability (HRV). Conversely, when a “relaxation” state is dominant, the sampling interval is widened to suppress battery consumption of the device. By this adaptive sampling control, the granularity of data collection is dynamically optimized, and efficient operation of system resources is realized without missing important physiological changes.

The acquisition unit can analyze past health data of the user and select an optimal acquisition method. The acquisition unit can, for example, analyze past heart rate data of the user and intensively acquire data in a time zone where an abnormality was observed. Also, the acquisition unit can analyze past sleep data of the user and optimize a data acquisition method during sleep. Furthermore, the acquisition unit can analyze past exercise data of the user and optimize a data acquisition method during exercise. Thereby, by analyzing past health data of the user and selecting an optimal acquisition method, efficient data acquisition becomes possible. Part or all of the above-described processing in the acquisition unit may be performed using AI or may be performed without using AI. Specifically, the acquisition unit implements a Reinforcement Learning agent and learns a policy (Policy) for determining a data acquisition action (when, which sensor, at what frequency to operate). This agent takes “current time, recent activity amount, past abnormality occurrence history” as a state space as input, and selects “sampling rate setting, sensor activation/sleep” as an action space. A reward function is designed based on a balance between “success of anomaly detection (positive reward)” and “battery consumption (negative reward)”. For example, when it is learned from past data that “the user tends to run from 18:00 to 19:00 on weekdays”, the agent selects an action of preloading (pre-activating) a GPS and a high-precision heart rate sensor immediately before that time zone, enabling acquisition of data without loss immediately after the start of exercise. In this way, by performing predictive resource management based on past history data, maximum monitoring performance is exhibited within a limited battery capacity.

The acquisition unit can perform filtering based on a current activity status or environment of the user when acquiring the health data. For example, when the user is exercising, the acquisition unit can acquire only data related to exercise. Also, when the user is resting, the acquisition unit can acquire only data related to rest. Furthermore, when the user is working, the acquisition unit can acquire only data related to work. Thereby, by filtering data based on the current activity status or environment of the user, highly relevant data can be acquired. Part or all of the above-described processing in the acquisition unit may be performed using AI or may be performed without using AI. Specifically, the acquisition unit executes a Human Activity Recognition (HAR) model taking time-series data from an acceleration sensor and a gyro sensor as input on an edge device (smartwatch etc.). This HAR model has a lightweight CNN or RNN structure and infers a current action class (e.g., stationary, walking, running, desk work, sleep) from input motion data in real time. The acquisition unit dynamically applies a data acquisition filter (mask) according to the inferred action class. For example, when the action class is “desk work”, acquisition of GPS data is stopped to protect privacy and power, while only an electrodermal activity (EDA) sensor for stress estimation and an acceleration sensor for posture detection are activated. On the other hand, in the case of the “running” class, the EDA sensor is turned off (because noise is superimposed due to sweating caused by exercise), and instead, the GPS and the heart rate sensor are recorded at the highest frequency. By this context-aware filtering processing, mixing of noise data in the subsequent analysis unit is prevented, and improvement in analysis accuracy and reduction in data traffic are simultaneously achieved.

The acquisition unit can estimate an emotion of the user and determine a priority of the health data to be acquired based on the estimated emotion of the user. For example, when the user is feeling stress, the acquisition unit can preferentially acquire data related to stress. Also, when the user is relaxed, the acquisition unit can preferentially acquire data related to relaxation. Furthermore, when the user is exercising, the acquisition unit can preferentially acquire data related to exercise. Thereby, by determining the priority of health data based on the emotion of the user, important data can be preferentially acquired. The estimation of emotion is realized using an emotion estimation function using, for example, an emotion engine or Generative AI. The Generative AI is text generation AI (for example, LLM), multimodal Generative AI, or the like, but is not limited to such examples. Part or all of the above-described processing in the acquisition unit may be performed using AI or may be performed without using AI. Specifically, the acquisition unit executes a scheduling algorithm that dynamically assigns a “priority score” to each sensor data stream. When the emotion estimation module outputs a “high stress” state (e.g., estimated from pitch fluctuation of voice or speech speed), the acquisition unit raises a priority score of R-R interval data (heart rate variability) related to autonomic nerve indices to the highest level, and preferentially transmits this data packet to a cloud server over other data (e.g., step count data) even when a communication bandwidth is tight (QoS control). Also, the acquisition unit adjusts sensitivity parameters of sensors and coefficients of noise removal filters according to the emotional state. For example, when it is estimated that the user is irritated (body movement is intense), the intensity of a motion artifact removal algorithm is increased to maintain signal quality. In this way, by dynamically reconfiguring the configuration of the data acquisition pipeline triggered by the internal state of emotion, data with the highest medical value at that moment is reliably captured.

The acquisition unit can preferentially acquire highly relevant data in consideration of geographical location information of the user when acquiring the health data. For example, when the user is at a high altitude, the acquisition unit can preferentially acquire health data related to high altitude. Also, when the user is in an urban area, the acquisition unit can preferentially acquire health data related to the urban area. Furthermore, when the user is at home, the acquisition unit can preferentially acquire health data related to home. Thereby, by acquiring data in consideration of the geographical location information of the user, highly relevant data can be preferentially acquired. Part or all of the above-described processing in the acquisition unit may be performed using AI or may be performed without using AI. Specifically, the acquisition unit links latitude/longitude/altitude data obtained from a GPS module with an external map database (GIS) or environmental API (weather, air pollution information). When the altitude data exceeds a threshold (for example, an altitude of 2000 m), the acquisition unit determines it as a “high altitude environment”, automatically increases an acquisition frequency of a blood oxygen saturation (SpO2) sensor, and shifts to a mode for monitoring a risk of hypoxia. Also, when the user is located in an area where an air pollution level in an urban area is high (an area with high PM2.5 concentration), the acquisition unit switches to a setting to preferentially perform detection of respiratory rate and cough (voice analysis by microphone input). Furthermore, when connection to a Wi-Fi access point at home is detected (geofencing), high-load sensing (GPS etc.) for outing is stopped, and the mode shifts to a minute body movement detection mode for measuring sleep quality. In this way, by combining location information and environmental context, intelligent data acquisition considering the influence of environmental factors on health is realized.

The acquisition unit can analyze social media activity of the user and acquire related data when acquiring the health data. For example, when the user is feeling stress on social media, the acquisition unit can acquire data related to stress. Also, when the user is relaxed on social media, the acquisition unit can acquire data related to relaxation. Furthermore, when the user is posting about exercise on social media, the acquisition unit can acquire data related to exercise. Thereby, by analyzing the social media activity of the user and acquiring data, highly relevant data can be acquired. Part or all of the above-described processing in the acquisition unit may be performed using AI or may be performed without using AI. Specifically, the acquisition unit periodically crawls posted texts and images of a social media account (SNS) linked based on the user's permission, and inputs them to a Natural Language Processing (NLP) model (for example, a sentiment analysis model using BERT or RoBERTa). This model extracts sentiment polarity (positive/negative) and specific keywords (e.g., “tired”, “at the gym”, “delicious”) from the posted content. For example, when the user posts posts containing negative emotions in succession late at night, the acquisition unit determines it as a sign of “mental instability” and activates a mode for recording sleep depth and nighttime heart rate variability in detail. Also, when the user posts a photo of running shoes, an image recognition model detects this and puts the GPS and the heart rate sensor into a standby state in preparation for data acquisition of exercise immediately after. In this way, by feeding back activity data in cyberspace to a sensing strategy in physical space, data collection adapted to the user's explicit and implicit states is enabled.

The analysis unit can estimate an emotion of the user and adjust a representation method of the analysis based on the estimated emotion of the user. For example, when the user is feeling stress, the analysis unit can provide a simple and highly visible analysis result. Also, when the user is relaxed, the analysis unit can provide a detailed analysis result. Furthermore, when the user is excited, the analysis unit can provide a visually stimulating analysis result. Thereby, by adjusting the representation method of the analysis based on the emotion of the user, an analysis result that is easy for the user to understand can be provided. The estimation of emotion is realized using an emotion estimation function using, for example, an emotion engine or Generative AI. The Generative AI is text generation AI (for example, LLM), multimodal Generative AI, or the like, but is not limited to such examples. Part or all of the above-described processing in the analysis unit may be performed using AI or may be performed without using AI. Specifically, the analysis unit includes a User Interface (UI) generation engine, and dynamically changes display parameters (color scheme, font size, information density, type of graph) based on an emotion estimation result. When it is determined that the user is in a “high stress” state, in order to minimize cognitive load, complex line graphs and detailed numerical tables are hidden, and instead, UI data of a “minimal mode” indicating the current health state only with simple icons such as “good” or “caution” and colors (green, yellow, red) is generated and output. On the other hand, when it is estimated that the user is “relaxed” and has high “inquisitiveness” (e.g., long stay time in the app), a UI of an “expert mode” including a power spectral density graph of heart rate variability and correlation analysis results for the past month is provided. This UI generation processing is optimized by a reinforcement learning model that takes the user's emotional state as input and outputs arrangement and attributes of UI components, and operates so as to maximize the user's engagement and understanding.

The analysis unit can adjust a level of detail of the analysis based on an importance of the health data during the analysis. For example, the analysis unit can perform detailed analysis on important health data. Also, the analysis unit can perform simplified analysis on health data with low importance. Furthermore, the analysis unit can determine a priority of the analysis according to the importance of the health data. Thereby, by adjusting the level of detail of the analysis based on the importance of the health data, efficient analysis becomes possible. Part or all of the above-described processing in the analysis unit may be performed using AI or may be performed without using AI. Specifically, the analysis unit adopts a two-stage configuration (cascade processing) of a lightweight first-stage model that performs “anomaly detection” in real time on an input data stream and a high-load second-stage model that performs detailed “cause identification” on a section where an abnormality is suspected. During normal times (data with low importance), only statistical processing with low calculation cost (check for deviation from moving average, etc.) is performed and completed on the edge device. However, when an important event (data with high importance) such as the heart rate exceeding a threshold is detected, a data window before and after that is cut out and transmitted to a high-performance GPU cluster on the cloud to execute detailed analysis by a large-scale Deep Neural Network (DNN). This DNN performs analysis at an advanced diagnostic support level, such as classification of types of arrhythmia and detection of signs of sleep apnea syndrome. In this way, by dynamically allocating calculation resources according to the medical importance of data, responsiveness and cost efficiency of the entire system are optimized.

The analysis unit can apply a different analysis algorithm depending on a category of the health data during the analysis. For example, the analysis unit can apply an analysis algorithm dedicated to heart rate for heart rate data. Also, the analysis unit can apply an analysis algorithm dedicated to sleep for sleep data. Furthermore, the analysis unit can apply an analysis algorithm dedicated to exercise for exercise data. Thereby, by applying a different analysis algorithm depending on the category of the health data, highly accurate analysis becomes possible. Part or all of the above-described processing in the analysis unit may be performed using AI or may be performed without using AI. Specifically, the analysis unit has a “model selector” function that selects and executes an appropriate inference model based on metadata (data type identifier) of input data. For heart rate data (PPG signal), a one-dimensional CNN or wavelet transform algorithm specialized for beat peak detection and noise removal is applied. For sleep data (acceleration+heart rate), an RNN-LSTM model or Hidden Markov Model (HMM) for determining a sleep stage (REM sleep, non-REM sleep, wakefulness) is applied. For exercise data (GPS+acceleration), a regression model (XGBoost etc.) for estimating exercise intensity (METs) and calorie consumption from a movement trajectory and speed change is applied. Furthermore, an ensemble learning layer that integrates outputs of these individual models is provided to execute cross-domain analysis, such as analyzing an influence of “lack of sleep (sleep model)” on “decline in exercise performance (exercise model)”.

The analysis unit can estimate an emotion of the user and adjust a length of the analysis based on the estimated emotion of the user. For example, when the user is in a hurry, the analysis unit can provide a short and concise analysis result. Also, when the user is relaxed, the analysis unit can provide a detailed analysis result. Furthermore, when the user is excited, the analysis unit can provide a visually stimulating analysis result. Thereby, by adjusting the length of the analysis based on the emotion of the user, an analysis result of a length appropriate for the user can be provided. The estimation of emotion is realized using an emotion estimation function using, for example, an emotion engine or Generative AI. The Generative AI is text generation AI (for example, LLM), multimodal Generative AI, or the like, but is not limited to such examples. Part or all of the above-described processing in the analysis unit may be performed using AI or may be performed without using AI. Specifically, the analysis unit includes a summarization function using a Large Language Model (LLM). The analysis unit takes a detailed analysis report (long text including numerical data and technical terms) as input, and inputs the user's emotional state and situation (context) to the LLM as a control code. When it is estimated that the user is in a state of “impatience” or “moving”, the LLM outputs a summary of a few words such as “Heart rate normal. No abnormality.” based on instructions of “Length constraint: Extremely short” and “Style: Conclusion only”. On the other hand, when the user is “relaxed” and at “home”, the LLM generates sentences of several paragraphs including meanings of respective indices and improvement advice based on instructions of “Length constraint: Detailed” and “Style: Explanatory”. By this variable length output generation, optimal information transmission adapted to the user's receptiveness is realized.

The analysis unit can determine a priority of the analysis based on an acquisition time of the health data during the analysis. For example, the analysis unit can preferentially analyze recently acquired health data. Also, the analysis unit can analyze current data while referring to past health data. Furthermore, the analysis unit can adjust the priority of the analysis according to the acquisition time of the health data. Thereby, by determining the priority of the analysis based on the acquisition time of the health data, efficient analysis becomes possible. Part or all of the above-described processing in the analysis unit may be performed using AI or may be performed without using AI. Specifically, the analysis unit implements a priority queuing mechanism (Priority Queue) that rearranges an order of a processing queue based on a timestamp of a data packet. Data within “the last 5 minutes” (hot data) for which real-time performance is required is sent to a low-latency in-memory processing pipeline, and anomaly detection is performed immediately. On the other hand, long-term trend analysis and periodic report generation processing using data of “one week ago” (cold data) are scheduled as batch processing jobs executed in the background during a time zone with low system load (for example, late at night). Also, when referring to past data in interpretation of current data, the analysis unit adopts a learning model using “Time Decay Weighting” that places more importance on recent data, and is designed to react sensitively to recent changes in physical condition.

The analysis unit can adjust an order of the analysis based on a relevance of the health data during the analysis. For example, the analysis unit can preferentially analyze highly relevant health data. Also, the analysis unit can analyze health data with low relevance later. Furthermore, the analysis unit can adjust the order of the analysis according to the relevance of the health data. Thereby, by adjusting the order of the analysis based on the relevance of the health data, efficient analysis becomes possible. Part or all of the above-described processing in the analysis unit may be performed using AI or may be performed without using AI. Specifically, the analysis unit uses a Graph Neural Network (GNN) or Bayesian network that models a causal relationship or correlation between different types of health data. For example, when an abnormality in heart rate is detected, the GNN identifies data of an “activity amount (exercise)” node or a “stress level” node that is strongly connected to a “heart rate” node, and dynamically raises analysis priorities of these data. Conversely, data analysis of “foot temperature” or the like having low relevance to the heart rate is postponed. In this way, by concentrating analysis resources based on a dependency structure (topology) between data, information necessary for Root Cause Analysis is processed in the shortest time, enabling rapid feedback.

The questioning unit can estimate an emotion of the user and adjust a representation method of the question based on the estimated emotion of the user. For example, when the user feels stressed, the questioning unit can ask a question using gentle wording. Also, when the user is relaxed, the questioning unit can ask a detailed question. Furthermore, when the user is in a hurry, the questioning unit can ask a concise question. Thereby, by adjusting the representation method of the question based on the user's emotion, it is possible to provide a question that is easy for the user to answer. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the questioning unit may be performed using AI or may be performed without using AI. Specifically, the questioning unit applies a style transfer technology in a generative AI (LLM). The questioning unit converts a writing style (tone & manner) in accordance with the user's emotional state while maintaining a core semantic content (semantics) of the question. For example, when a basic question is “What were you doing?”, if it is estimated that the user is in a state of “sadness” or “depression”, the LLM is driven by parameters such as “Persona: Counselor” and “Tone: Empathetic/Receptive”, and rewrites it into a considerate expression such as “If you don't mind, could you tell me what the situation was like at that time? You don't have to answer if you don't want to.” Conversely, when the user is “active” and “positive”, a friendly and short expression such as “What were you doing now? Tell me!” is selected. This emotion-adaptive dialogue generation forms a rapport (trust relationship) with the user and improves a response rate and quality of answers.

The questioning unit can refer to a past answer history of the user and select an optimal question during questioning. For example, the questioning unit can ask a related question based on content answered by the user in the past. Also, the questioning unit can select an optimal question from the past answer history of the user. Furthermore, the questioning unit can analyze the past answer history of the user and ask an effective question. Thereby, by referring to the past answer history of the user, it is possible to provide a highly relevant question. Part or all of the above-described processing in the questioning unit may be performed using AI or may be performed without using AI. Specifically, the questioning unit adopts a vector database that vectorizes and stores past dialogue logs with the user, and a Retrieval-Augmented Generation (RAG) architecture. When generating a question, the questioning unit searches the database for past dialogue cases similar to a current situation (query vector). For example, if a history is found where the user answered “I was nervous before a presentation” during a similar increase in heart rate in the past, the LLM incorporates that information as context and generates a personalized question based on memory, such as “Are you nervous before a work presentation again this time?”. Thereby, the user feels that “the system understands me”, and the trouble of explaining the situation from scratch is saved, so that user experience (UX) is significantly improved.

The questioning unit can customize content of the question based on a current situation of the user during questioning. For example, when the user is exercising, the questioning unit can ask a question related to exercise. Also, when the user is resting, the questioning unit can ask a question related to rest. Furthermore, when the user is working, the questioning unit can ask a question related to work. Thereby, by customizing the content of the question based on the current situation of the user, it is possible to provide an appropriate question. Part or all of the above-described processing in the questioning unit may be performed using AI or may be performed without using AI. Specifically, the questioning unit uses a template engine or an LLM that receives context labels estimated from sensor data (e.g., [Activity: Running], [Location: Park], [Weather: Sunny]) as input variables. When the user is running (heart rate is high and GPS is moving), the questioning unit suppresses “questions requiring stopping and operating” and generates a simple question that can be answered with a binary answer, such as “Is the current pace not too hard? (Yes/No)”, via a voice interface. On the other hand, when the user is resting at home (heart rate is stable, location is home), the questioning unit displays a free-description type question form such as “Please tell me in detail about your physical condition today” on a screen of a smartphone. In this way, not only the content of the question but also a modality of the question (voice, text, touch operation) and a depth of dialogue are dynamically optimized according to the user's physical and environmental situation.

The questioning unit can estimate an emotion of the user and determine a priority of the question based on the estimated emotion of the user. For example, when the user feels stressed, the questioning unit can preferentially ask a question related to stress. Also, when the user is relaxed, the questioning unit can preferentially ask a question related to relaxing. Furthermore, when the user is exercising, the questioning unit can preferentially ask a question related to exercise. Thereby, by determining the priority of the question based on the user's emotion, it is possible to preferentially ask an important question. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the questioning unit may be performed using AI or may be performed without using AI. Specifically, the questioning unit executes a ranking algorithm that calculates a relevance score (cosine similarity, etc.) with a current user's emotion state vector for a plurality of question candidates (e.g., Q1 “What is the meal content?”, Q2 “Is sleep sufficient?”, Q3 “Do you have any worries?”). When the user shows “anxiety” or “high stress”, the score of Q3 related to mental health becomes high and is placed at the top of a list. Conversely, when the user is full of “vitality”, a question regarding activity amount or performance improvement is prioritized. Furthermore, the questioning unit estimates the user's emotional margin (cognitive bandwidth), and if there is no margin, performs filtering processing to ask only the question with the first priority and discard or postpone the others.

The questioning unit can provide an optimal question in consideration of geographical location information of the user during questioning. For example, when the user is at a high altitude, the questioning unit can ask a question related to high altitude. Also, when the user is in an urban area, the questioning unit can ask a question related to the urban area. Furthermore, when the user is at home, the questioning unit can ask a question related to home. Thereby, by providing the question in consideration of the geographical location information of the user, it is possible to ask a highly relevant question. Part or all of the above-described processing in the questioning unit may be performed using AI or may be performed without using AI. Specifically, the questioning unit incorporates semantic information of a place (POI: Point of Interest, e.g., “hospital”, “gym”, “office”, “mountainous area”) obtained by reverse geocoding GPS coordinates into a prompt for question generation. When it is detected that the user is at a “hospital”, the questioning unit stops normal health check questions and switches to a support-type question suitable for the place, such as “Do you want to keep a record of the medical examination?” or “Do you want to take a note of what you want to ask the doctor?”. Also, when the user is at a high altitude, a specific question confirming hypoxic symptoms such as “Do you feel shortness of breath?” is generated. In this way, the location information is utilized not merely as coordinates but as a context suggesting the user's behavioral intention or environmental risk.

The questioning unit can analyze social media activity of the user and ask a related question during questioning. For example, when the user feels stressed on social media, the questioning unit can ask a question related to stress. Also, when the user is relaxed on social media, the questioning unit can ask a question related to relaxing. Furthermore, when the user posts about exercise on social media, the questioning unit can ask a question related to exercise. Thereby, by analyzing the social media activity of the user and asking the question, it is possible to provide a highly relevant question. Part or all of the above-described processing in the questioning unit may be performed using AI or may be performed without using AI. Specifically, the questioning unit uses a topic model (topic distribution by LDA, etc.) extracted from posted content on SNS or an emotion analysis result as an input parameter of a question generation engine. For example, when the user has posted “Work never ends” on SNS, the questioning unit catches the information and generates a question based on the context on SNS, such as “How is the progress of your work? Would you like to take a short break?”, when detecting an increase in heart rate. This complements a social and psychological background that cannot be grasped only by sensor data, and realizes a natural dialogue “with context” for the user.

The advice unit can estimate an emotion of the user and adjust a representation method of the advice based on the estimated emotion of the user. For example, when the user feels stressed, the advice unit can provide advice using gentle wording. Also, when the user is relaxed, the advice unit can provide detailed advice. Furthermore, when the user is in a hurry, the advice unit can provide concise advice. Thereby, by adjusting the representation method of the advice based on the user's emotion, it is possible to provide advice that is easy for the user to accept. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the advice unit may be performed using AI or may be performed without using AI. Specifically, the advice unit has an NLG (Natural Language Generation) module that converts an intermediate representation describing a recommended health action (action) (e.g., {action: “deep_breath”, duration: “5 min”}) into natural language according to the user's emotional state. When the user is in a state of “anger” or “irritation”, the NLG module avoids an imperative form and selects a proposal type (soft expression) such as “Taking a deep breath might calm you down a little”. On the other hand, when the user feels a “sense of accomplishment” (such as after exercise), an energetic expression including praise such as “Great! Let's do a cool-down in this state” is selected. This expression adjustment according to the emotion increases the user's acceptance (compliance) of the advice and promotes behavior change.

The advice unit can analyze past health data of the user and provide optimal advice when providing advice. For example, the advice unit can analyze past heart rate data of the user and advise on a management method of the heart rate. Also, the advice unit can analyze past sleep data of the user and advise on an improvement method of sleep. Furthermore, the advice unit can analyze past exercise data of the user and advise on an effective method of exercise. Thereby, by analyzing the past health data of the user and providing the advice, it is possible to provide highly relevant advice. Part or all of the above-described processing in the advice unit may be performed using AI or may be performed without using AI. Specifically, the advice unit refers to a database of “action-result pairs” recording the user's past actions and changes in health indicators corresponding thereto, and selects an intervention method with the highest effect using a causal inference model or reinforcement learning (bandit algorithm, etc.). For example, if there is data that sleep quality (ratio of deep sleep) improved on a day when “stretching before going to bed” was proposed in the past, and there was no change on a day when “herbal tea” was proposed, the advice unit preferentially recommends “stretching” for which a statistically significant improvement effect was observed. In this way, by generating evidence-based advice based on the user's own data (experimental result of N=1) rather than a general theory, an optimal solution matching an individual constitution and lifestyle is provided.

The advice unit can customize content of the advice based on a current living situation of the user when providing advice. For example, when the user is exercising, the advice unit can provide advice related to exercise. Also, when the user is resting, the advice unit can provide advice related to rest. Furthermore, when the user is working, the advice unit can provide advice related to work. Thereby, by customizing the content of the advice based on the current living situation of the user, it is possible to provide appropriate advice. Part or all of the above-described processing in the advice unit may be performed using AI or may be performed without using AI. Specifically, the advice unit uses a rule engine or an AI model that filters advice with high feasibility based on a real-time context recognition result. For example, when the user is “in a meeting (determined by calendar linkage or voice analysis)”, advice such as “stand up and take a walk” is excluded because it is difficult to execute even if a stress value is high, and instead, a micro-action executable on the spot such as “take a deep breath inconspicuously” or “correct posture” is proposed. Also, when the user is “in a commuter train”, situation-specific advice such as “train the trunk using a strap” is provided. This enables natural incorporation of health actions without interrupting the flow of the user's life.

The advice unit can estimate an emotion of the user and determine a priority of the advice based on the estimated emotion of the user. For example, when the user feels stressed, the advice unit can preferentially provide advice on stress management. Also, when the user is relaxed, the advice unit can preferentially provide advice related to relaxing. Furthermore, when the user is exercising, the advice unit can preferentially provide advice related to exercise. Thereby, by determining the priority of the advice based on the user's emotion, it is possible to preferentially provide important advice. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the advice unit may be performed using AI or may be performed without using AI. Specifically, the advice unit implements a prioritization logic applying Maslow's hierarchy of needs or a behavior change stage model. When the user feels strong “anxiety” or “pain” (a state where safety needs are threatened), the advice unit temporarily stops advice regarding a long-term health goal (diet, etc.) and provides a “coping strategy” that brings immediate mental stability with the highest priority. Conversely, in a state where the emotion is stable and motivation is high, the priority of advice close to “self-actualization” such as high-load exercise or dietary restriction is raised. In this way, by adjusting a level of intervention according to availability of the user's psychological resources, pushiness of the advice is eliminated and a continuation rate is increased.

The advice unit can provide optimal advice in consideration of geographical location information of the user when providing advice. For example, when the user is at a high altitude, the advice unit can provide advice related to high altitude. Also, when the user is in an urban area, the advice unit can provide advice related to the urban area. Furthermore, when the user is at home, the advice unit can provide advice related to home. Thereby, by providing the advice in consideration of the geographical location information of the user, it is possible to provide highly relevant advice. Part or all of the above-described processing in the advice unit may be performed using AI or may be performed without using AI. Specifically, the advice unit refers to an environmental database (weather, temperature, humidity, pollen scattering amount, noise level, etc.) linked with the location information. For example, when the user is in a place with high temperature and low humidity, the advice unit calculates a “risk of dehydration” and sends a notification urging hydration more frequently than usual. Also, when the user is near a park or a green space, the advice unit proposes a walking route in that place in order to maximize a “forest bathing effect”. Furthermore, detecting a timing of returning home, the advice unit triggers a health action embedded in a context of place, such as proposing a reminder for “hand washing and gargling” or “changing into room wear (switching to relax mode)”.

The advice unit can analyze social media activity of the user and provide related advice when providing advice. For example, when the user feels stressed on social media, the advice unit can provide advice on stress management. Also, when the user is relaxed on social media, the advice unit can provide advice related to relaxing. Furthermore, when the user posts about exercise on social media, the advice unit can provide advice related to exercise. Thereby, by analyzing the social media activity of the user and providing the advice, it is possible to provide highly relevant advice. Part or all of the above-described processing in the advice unit may be performed using AI or may be performed without using AI. Specifically, the advice unit analyzes a social graph on SNS or a trend of a community, and generates advice using a principle of social proof. For example, when a friend of the user posts a result of running and the user himself/herself shows interest in exercise, the advice unit generates a message including social motivation, such as “Mr./Ms. XX, your friend, is also running. Why don't you go for a light jog too?”. Also, the advice unit detects a keyword regarding a health method or a recipe popular on SNS, and if it is medically appropriate, proposes “How about the popular XX recipe for today's dinner?”. In this way, by utilizing not only individual data but also social connections and trend information, the user's motivation is stimulated from the outside.

The system according to the embodiment is not limited to the above-described examples, and various modifications are possible, for example, as follows. Specifically, the present system can be implemented not only in a cloud computing environment but also in an edge computing environment (on-device AI) or a hybrid configuration thereof. Also, each functional unit (acquisition unit, analysis unit, questioning unit, advice unit) may be configured as a software module operating on a single processor, or may be configured as a network-distributed microservice architecture. Furthermore, the AI model used is not limited to a pre-trained fixed model, but may be an online learning model updated daily based on user feedback, or a federated learning model that integrates and learns data of a plurality of users while protecting privacy.

The acquisition unit can estimate an emotion of the user and adjust an acquisition timing of the health data based on the estimated emotion of the user. For example, when the user feels stressed, an acquisition frequency of the health data can be increased until the stress is alleviated. Also, when the user is relaxed, the acquisition frequency of the health data can be decreased to reduce a burden on the user. Furthermore, when the user is exercising, the health data can be acquired at a peak of the exercise to collect detailed data. Thereby, by adjusting the acquisition timing of the health data based on the user's emotion, it is possible to acquire more appropriate data. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the acquisition unit may be performed using AI or may be performed without using AI. As a modification, the acquisition unit can adopt a configuration in which a lightweight emotion estimation model (quantized neural network, etc.) is mounted in an edge device such as a smart watch, and the emotion is determined in real time without communication delay to control the sensor. In this case, by controlling the data transmission frequency to the cloud itself based on the emotion (e.g., not transmitting during calm times, transmitting only during emotional ups and downs), both privacy protection and communication cost reduction are achieved.

The analysis unit can estimate an emotion of the user and adjust a representation method of the analysis based on the estimated emotion of the user. For example, when the user feels stressed, a simple and highly visible analysis result can be provided. Also, when the user is relaxed, a detailed analysis result can be provided. Furthermore, when the user is excited, a visually stimulating analysis result can be provided. Thereby, by adjusting the representation method of the analysis based on the user's emotion, it is possible to provide an analysis result that is easy for the user to understand. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the analysis unit may be performed using AI or may be performed without using AI. As a modification, the analysis unit can cooperate with a display device such as augmented reality (AR) glasses or a smart mirror to perform immersive analysis result display such as a 3D graph floating in space or an explanation by an avatar according to the user's emotional state. For example, when the user is depressed, the analysis result is gently presented together with an animation in which the avatar snuggles up.

The questioning unit can estimate an emotion of the user and adjust a representation method of the question based on the estimated emotion of the user. For example, when the user feels stressed, the questioning unit can ask a question using gentle wording. Also, when the user is relaxed, the questioning unit can ask a detailed question. Furthermore, when the user is in a hurry, the questioning unit can ask a concise question. Thereby, by adjusting the representation method of the question based on the user's emotion, it is possible to provide a question that is easy for the user to answer. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the questioning unit may be performed using AI or may be performed without using AI. As a modification, the questioning unit has a function of dynamically changing parameters (voice quality, intonation, speed) of a voice synthesis AI (Text-to-Speech) according to the emotion. When the user is tired, the question is asked slowly with a low and calm voice, and when the user is energetic, the question is asked with a bright and up-tempo voice, thereby giving consideration to the user also from an auditory aspect.

The advice unit can estimate an emotion of the user and adjust a representation method of the advice based on the estimated emotion of the user. For example, when the user feels stressed, the advice unit can provide advice using gentle wording. Also, when the user is relaxed, the advice unit can provide detailed advice. Furthermore, when the user is in a hurry, the advice unit can provide concise advice. Thereby, by adjusting the representation method of the advice based on the user's emotion, it is possible to provide advice that is easy for the user to accept. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the advice unit may be performed using AI or may be performed without using AI. As a modification, the advice unit can cooperate with a music generation AI to adopt a configuration in which BGM or environmental sound (e.g., sound of rain or forest sound at the time of relaxation advice) matching the content of the advice or the user's emotion is reproduced simultaneously, thereby auditorily amplifying the effect of the advice.

The advice unit can estimate an emotion of the user and determine a priority of the advice based on the estimated emotion of the user. For example, when the user feels stressed, the advice unit can preferentially provide advice on stress management. Also, when the user is relaxed, the advice unit can preferentially provide advice related to relaxing. Furthermore, when the user is exercising, the advice unit can preferentially provide advice related to exercise. Thereby, by determining the priority of the advice based on the user's emotion, it is possible to preferentially provide important advice. The estimation of the emotion is realized using an emotion estimation function using, for example, an emotion engine or a generative AI. The generative AI is a text generation AI (for example, LLM) or a multimodal generative AI, but is not limited to such examples. Part or all of the above-described processing in the advice unit may be performed using AI or may be performed without using AI. As a modification, the advice unit can adopt a configuration in which a council system of a plurality of expert AI agents (nutritionist agent, trainer agent, mental coach agent) is adopted, and which agent's proposal should be adopted in the current user's emotional state is determined by a meta-learning model.

The acquisition unit can analyze past health data of the user and select an optimal acquisition method. For example, the acquisition unit can analyze past heart rate data of the user and intensively acquire data in a time zone where an abnormality was observed. Also, the acquisition unit can analyze past sleep data of the user and optimize a data acquisition method during sleep. Furthermore, the acquisition unit can analyze past exercise data of the user and optimize a data acquisition method during exercise. Thereby, by analyzing the past health data of the user and selecting the optimal acquisition method, efficient data acquisition becomes possible. Part or all of the above-described processing in the acquisition unit may be performed using AI or may be performed without using AI. As a modification, the acquisition unit can anonymize and analyze data of other user groups (cohorts) having similar attributes (age, gender, lifestyle habits) as well as the user's individual data, and learn a time zone or pattern of health risks frequently occurring in the cohort, thereby applying an optimized data acquisition schedule (cold start countermeasure) from an initial stage even for a new user.

The acquisition unit can perform filtering based on a current activity status or environment of the user when acquiring the health data. For example, when the user is exercising, only data related to exercise can be acquired. Also, when the user is resting, only data related to rest can be acquired. Furthermore, when the user is working, only data related to work can be acquired. Thereby, by filtering the data based on the current activity status or environment of the user, it is possible to acquire highly relevant data. Part or all of the above-described processing in the acquisition unit may be performed using AI or may be performed without using AI. As a modification, the acquisition unit can utilize filtering at a sensor hardware level (for example, interrupt generation by threshold determination in a FIFO buffer in an acceleration sensor) to implement an ultra-low power consumption mode in which the system is woken up to acquire data only at the moment when a specific activity pattern (e.g., fall, severe collision) occurs while keeping a main processor in sleep.

The analysis unit can adjust a level of detail of the analysis based on an importance of the health data during the analysis. For example, detailed analysis can be performed on important health data. Also, the analysis unit can perform simplified analysis on health data with low importance. Furthermore, the analysis unit can determine a priority of the analysis according to the importance of the health data. Thereby, by adjusting the level of detail of the analysis based on the importance of the health data, efficient analysis becomes possible. Part or all of the above-described processing in the analysis unit may be performed using AI, or may be performed without using AI. As a modification, the analysis unit can perform “offloading” control to dynamically switch a location where processing is executed according to the importance of the data. Processing with low importance that requires immediacy is completed within a smartwatch, and only processing with high importance that requires computational resources (e.g., disease diagnosis by waveform analysis of an electrocardiogram) is offloaded to a cloud server via 5G communication, thereby optimizing a balance between delay and accuracy.

The questioning unit can refer to a past answer history of the user and select an optimal question during questioning. For example, based on content answered by the user in the past, a related question can be asked. Also, the questioning unit can select an optimal question from the past answer history of the user. Furthermore, the questioning unit can analyze the past answer history of the user and ask an effective question. Thereby, by referring to the past answer history of the user, a highly relevant question can be provided. Part or all of the above-described processing in the questioning unit may be performed using AI, or may be performed without using AI. As a modification, the questioning unit can be equipped with an adaptive interface that models user answer tendencies (honesty, detail, answer delay time) and learns timings and question formats (yes/no, 5-point scale, voice input) that are easy for the user to answer. This efficiently collects necessary information while minimizing a burden on the user.

The advice unit can analyze past health data of the user and provide optimal advice when providing advice. For example, past heart rate data of the user can be analyzed to advise on a method for managing heart rate. Also, the advice unit can analyze past sleep data of the user and advise on a method for improving sleep. Furthermore, the advice unit can analyze past exercise data of the user and advise on an effective method of exercise. Thereby, by analyzing the past health data of the user and providing advice, highly relevant advice can be given. Part or all of the above-described processing in the advice unit may be performed using AI, or may be performed without using AI. As a modification, the advice unit can have a self-improvement function that performs counterfactual analysis to simulate “what the health condition would have been if this advice had been followed in the past” and corrects a future advice strategy based on a result thereof. This realizes a system that eliminates ineffective advice and continues to evolve into more effective advice.

A flow of processing of Example of the Embodiment will be briefly described below. This processing flow is a series of sequences realized by a hardware processor executing a program stored in a memory, and each step is executed synchronously or asynchronously.

Step 1: The acquisition unit acquires health data. The health data includes heart rate, blood pressure, body temperature, and the like. The acquisition unit acquires data from a sensor of a smartwatch, a smartphone of the user, or another wearable device. Specifically, the acquisition unit establishes a connection with each sensor device, starts stream reception of biological signals at a predetermined sampling rate (e.g., 1 Hz to 100 Hz), and stores received data in a temporary buffer. Step 2: The analysis unit analyzes the data acquired by the acquisition unit and detects an abnormal numerical value or pattern. The analysis unit analyzes heart rate data and detects an abnormal pattern (for example, a rapid fluctuation in heart rate or a continuous high heart rate). The analysis unit analyzes the data using AI. Specifically, the analysis unit reads data from the buffer, performs preprocessing, and then inputs the data into a trained neural network to calculate an anomaly score, and issues an event detection signal when the score exceeds a threshold. Step 3: The questioning unit asks the user a question based on the abnormal numerical value or pattern detected by the analysis unit. The questioning unit asks the user what event occurred at that time. A format of the question includes a selection type, a free description type, and the like. The questioning unit asks the user a question using AI. Specifically, upon receiving the event detection signal, the questioning unit generates a question sentence according to a situation using an LLM, outputs the question sentence to the user through a display or a speaker of a user terminal, and waits for an input from the user. Step 4: The advice unit identifies a cause based on an answer of the user obtained by the questioning unit and provides appropriate advice. The advice unit proposes a breathing method or a stretching method for relaxing, or an improvement point regarding meal content or meal timing. The advice unit provides appropriate advice to the user using AI. Specifically, the advice unit infers a cause by integrating a sensor analysis result and the user answer, searches for or generates optimal countermeasure content from a database to present to the user, and monitors a subsequent biological reaction (feedback) to verify an effect of the advice.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of a plurality of elements including the above-described acquisition unit, analysis unit, questioning unit, and advice unit is realized by, for example, at least one of a smart device 14 and a data processing device 12. For example, the acquisition unit acquires health data using a sensor of the smart device 14. Also, the analysis unit is realized by a specific processing unit 290 of the data processing device 12, analyzes the acquired data, and detects an abnormal numerical value or pattern. The questioning unit is realized by, for example, a control unit 46A of the smart device 14, and asks the user a question. The advice unit is realized by, for example, the specific processing unit 290 of the data processing device 12, and provides appropriate advice to the user. The correspondence relationship between each unit and the device or control unit is not limited to the above-described example, and various modifications are possible.

### Second Embodiment

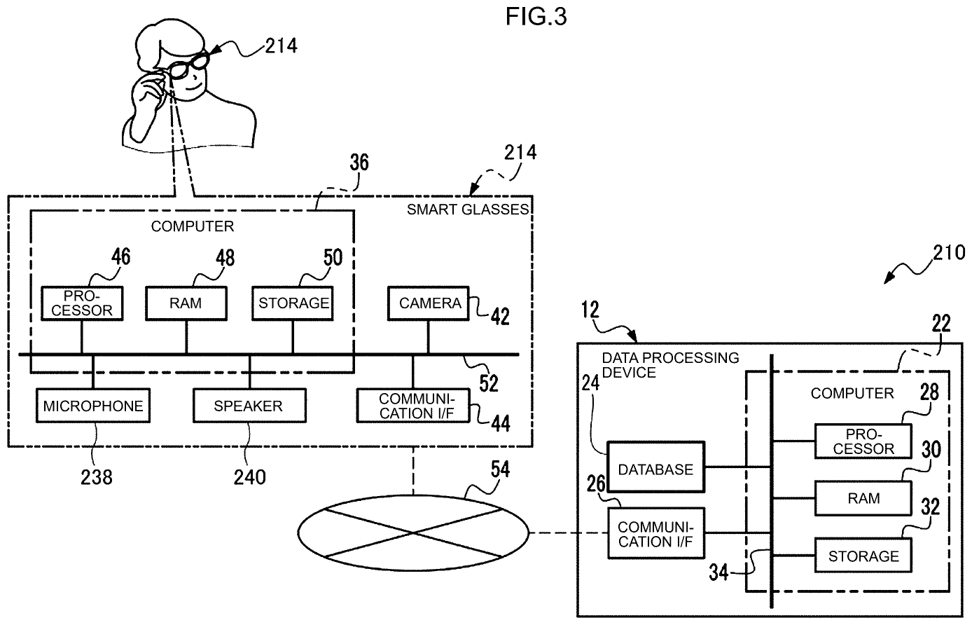

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

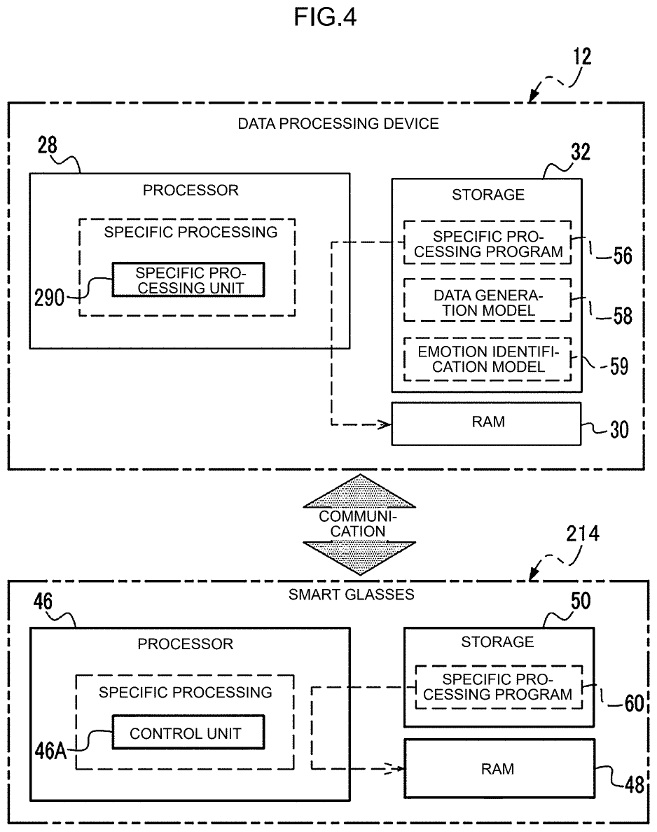

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of a plurality of elements including the above-described acquisition unit, analysis unit, questioning unit, and advice unit is realized by, for example, at least one of smart glasses 214 and the data processing device 12. For example, the acquisition unit acquires health data using a sensor of the smart glasses 214. Also, the analysis unit is realized by the specific processing unit 290 of the data processing device 12, analyzes the acquired data, and detects an abnormal numerical value or pattern. The questioning unit is realized by, for example, a control unit 46A of the smart glasses 214, and asks the user a question. The advice unit is realized by, for example, the specific processing unit 290 of the data processing device 12, and provides appropriate advice to the user. The correspondence relationship between each unit and the device or control unit is not limited to the above-described example, and various modifications are possible.

### Third Embodiment

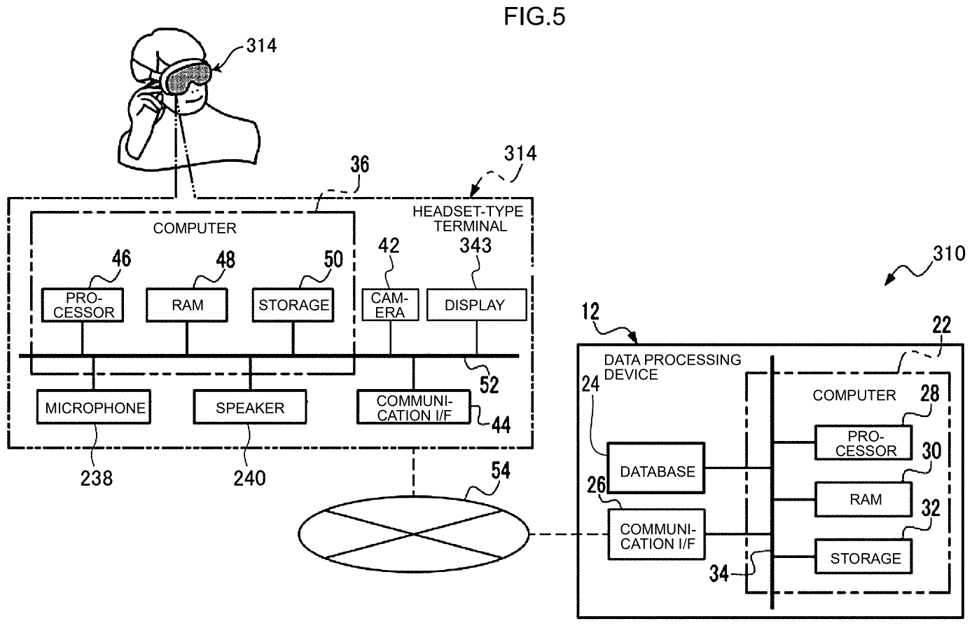

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

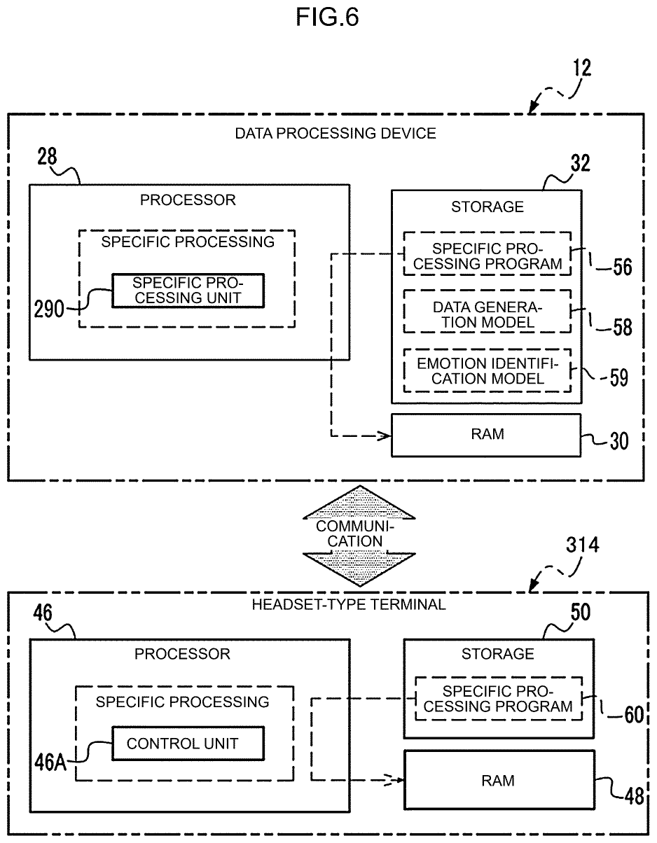

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of a plurality of elements including the above-described acquisition unit, analysis unit, questioning unit, and advice unit is realized by, for example, at least one of a headset-type terminal 314 and the data processing device 12. For example, the acquisition unit acquires health data using a sensor of the headset-type terminal 314. Also, the analysis unit is realized by the specific processing unit 290 of the data processing device 12, analyzes the acquired data, and detects an abnormal numerical value or pattern. The questioning unit is realized by, for example, a control unit 46A of the headset-type terminal 314, and asks the user a question. The advice unit is realized by, for example, the specific processing unit 290 of the data processing device 12, and provides appropriate advice to the user. The correspondence relationship between each unit and the device or control unit is not limited to the above-described example, and various modifications are possible.

### Fourth Embodiment

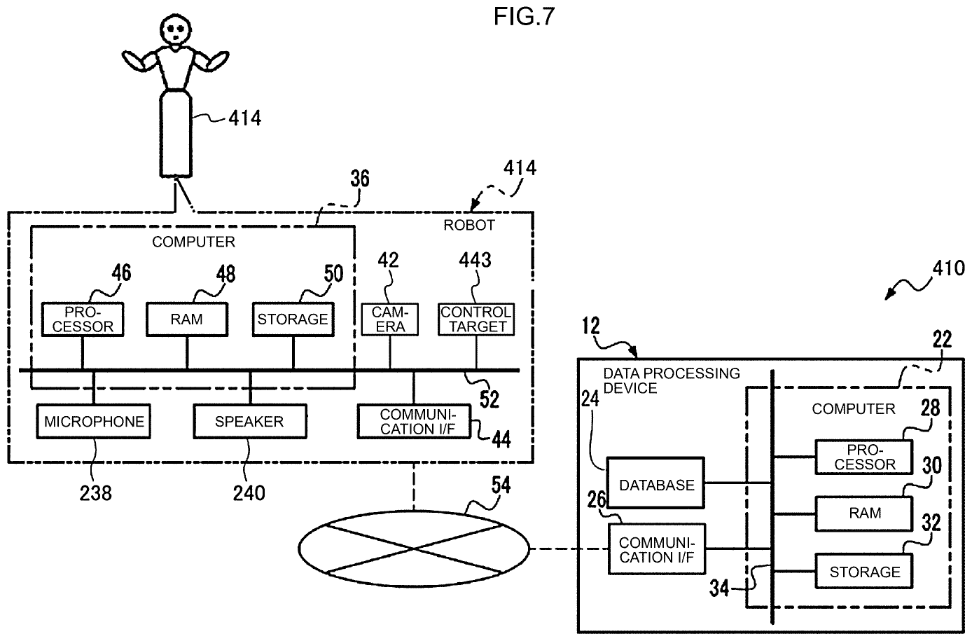

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

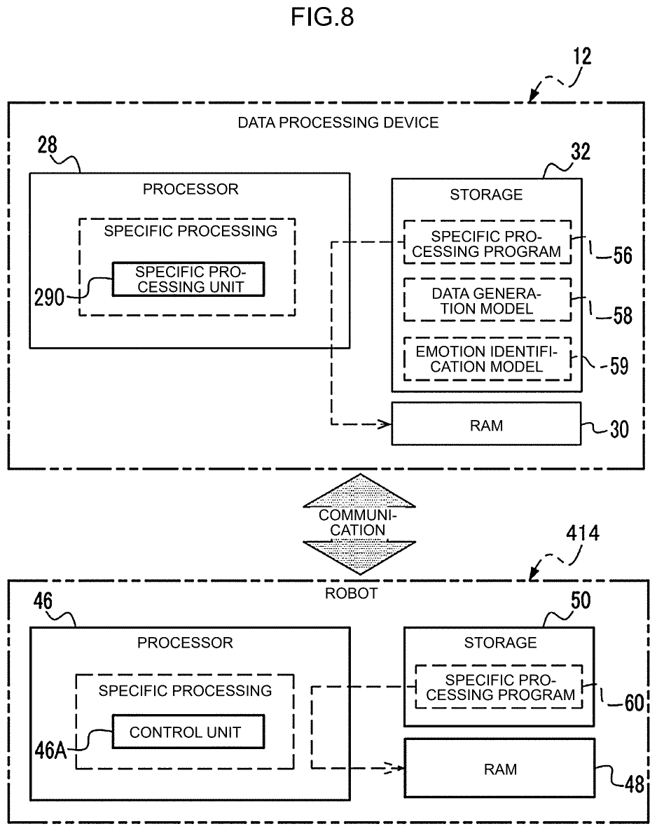

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of a plurality of elements including the above-described acquisition unit, analysis unit, questioning unit, and advice unit is realized by, for example, at least one of a robot 414 and the data processing device 12. For example, the acquisition unit acquires health data using a sensor of the robot 414. Also, the analysis unit is realized by the specific processing unit 290 of the data processing device 12, analyzes the acquired data, and detects an abnormal numerical value or pattern. The questioning unit is realized by, for example, a control unit 46A of the robot 414, and asks the user a question. The advice unit is realized by, for example, the specific processing unit 290 of the data processing device 12, and provides appropriate advice to the user. The correspondence relationship between each unit and the device or control unit is not limited to the above-described example, and various modifications are possible.

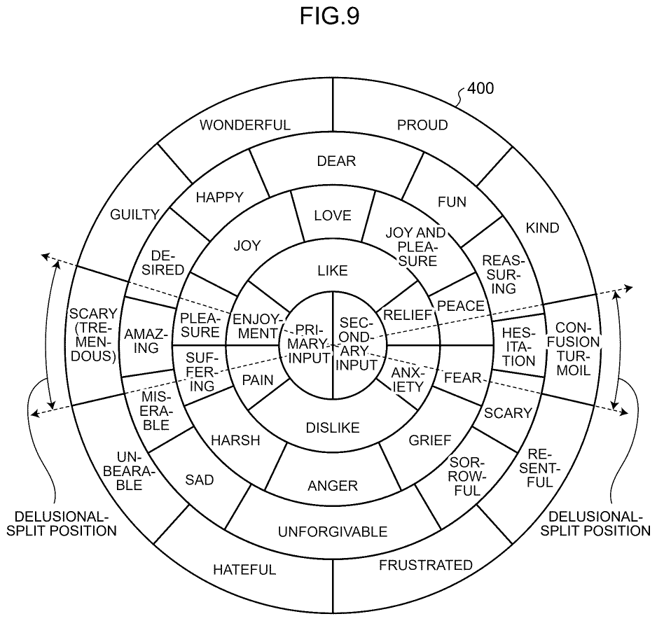

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

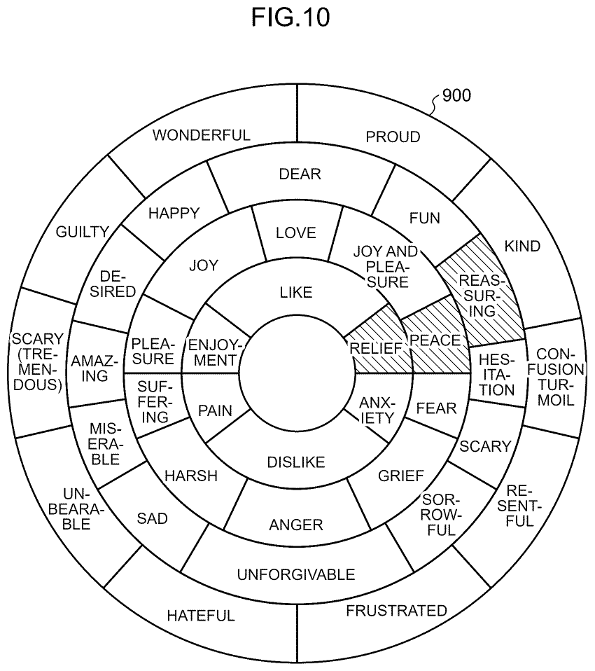

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.

(Supplementary Note 1) A system comprising: an acquisition unit configured to acquire health data; an analysis unit configured to analyze the data acquired by the acquisition unit and detect an abnormal numerical value or pattern; a questioning unit configured to ask a user a question based on the abnormal numerical value or pattern detected by the analysis unit; and an advice unit configured to identify a cause based on an answer of the user obtained by the questioning unit and provide advice.

(Supplementary Note 2) The system according to Supplementary Note 1, wherein the advice unit is configured to propose a breathing method or a stretching method for relaxing.

(Supplementary Note 3) The system according to Supplementary Note 1, wherein the advice unit is configured to propose an improvement point regarding meal content or meal timing.

(Supplementary Note 4) The system according to Supplementary Note 1, wherein the analysis unit is configured to analyze heart rate data and detect an abnormal pattern.

(Supplementary Note 5) The system according to Supplementary Note 1, wherein the questioning unit is configured to ask the user what event occurred at that time.

(Supplementary Note 6) The system according to Supplementary Note 1, wherein the advice unit is configured to recommend a review of lifestyle habits or a medical examination by a doctor when a spike in heart rate is observed periodically.

(Supplementary Note 7) The system according to Supplementary Note 1, wherein the acquisition unit is configured to estimate an emotion of the user and adjust an acquisition timing of the health data based on the estimated emotion of the user.

(Supplementary Note 8) The system according to Supplementary Note 1, wherein the acquisition unit is configured to analyze past health data of the user and select an optimal acquisition method.

(Supplementary Note 9) The system according to Supplementary Note 1, wherein the acquisition unit is configured to perform filtering based on a current activity status or environment of the user when acquiring the health data.

(Supplementary Note 10) The system according to Supplementary Note 1, wherein the acquisition unit is configured to estimate an emotion of the user and determine a priority of the health data to be acquired based on the estimated emotion of the user.

(Supplementary Note 11) The system according to Supplementary Note 1, wherein the acquisition unit is configured to preferentially acquire highly relevant data in consideration of geographical location information of the user when acquiring the health data.

(Supplementary Note 12) The system according to Supplementary Note 1, wherein the acquisition unit is configured to analyze social media activity of the user and acquire related data when acquiring the health data.

(Supplementary Note 13) The system according to Supplementary Note 1, wherein the analysis unit is configured to estimate an emotion of the user and adjust a representation method of the analysis based on the estimated emotion of the user.

(Supplementary Note 14) The system according to Supplementary Note 1, wherein the analysis unit is configured to adjust a level of detail of the analysis based on an importance of the health data during the analysis.

(Supplementary Note 15) The system according to Supplementary Note 1, wherein the analysis unit is configured to apply a different analysis algorithm depending on a category of the health data during the analysis.

(Supplementary Note 16) The system according to Supplementary Note 1, wherein the analysis unit is configured to estimate an emotion of the user and adjust a length of the analysis based on the estimated emotion of the user.

(Supplementary Note 17) The system according to Supplementary Note 1, wherein the analysis unit is configured to determine a priority of the analysis based on an acquisition time of the health data during the analysis.

(Supplementary Note 18) The system according to Supplementary Note 1, wherein the analysis unit is configured to adjust an order of the analysis based on a relevance of the health data during the analysis.

(Supplementary Note 19) The system according to Supplementary Note 1, wherein the questioning unit is configured to estimate an emotion of the user and adjust a representation method of the question based on the estimated emotion of the user.

(Supplementary Note 20) The system according to Supplementary Note 1, wherein the questioning unit is configured to refer to a past answer history of the user and select an optimal question during questioning.

(Supplementary Note 21) The system according to Supplementary Note 1, wherein the questioning unit is configured to customize content of the question based on a current situation of the user during questioning.

(Supplementary Note 22) The system according to Supplementary Note 1, wherein the questioning unit is configured to estimate an emotion of the user and determine a priority of the question based on the estimated emotion of the user.

(Supplementary Note 23) The system according to Supplementary Note 1, wherein the questioning unit is configured to provide an optimal question in consideration of geographical location information of the user during questioning.

(Supplementary Note 24) The system according to Supplementary Note 1, wherein the questioning unit is configured to analyze social media activity of the user and ask a related question during questioning.

(Supplementary Note 25) The system according to Supplementary Note 1, wherein the advice unit is configured to estimate an emotion of the user and adjust a representation method of the advice based on the estimated emotion of the user.

(Supplementary Note 26) The system according to Supplementary Note 1, wherein the advice unit is configured to analyze past health data of the user and provide optimal advice when providing advice.

(Supplementary Note 27) The system according to Supplementary Note 1, wherein the advice unit is configured to customize content of the advice based on a current living situation of the user when providing advice.

(Supplementary Note 28) The system according to Supplementary Note 1, wherein the advice unit is configured to estimate an emotion of the user and determine a priority of the advice based on the estimated emotion of the user.

(Supplementary Note 29) The system according to Supplementary Note 1, wherein the advice unit is configured to provide optimal advice in consideration of geographical location information of the user when providing advice.

(Supplementary Note 30) The system according to Supplementary Note 1, wherein the advice unit is configured to analyze social media activity of the user and provide related advice when providing advice.

What is claimed is:

1. A system comprising:
a communication interface configured to communicate with a client terminal via a packet-switched network;
a memory storing a data generation model obtained by deep learning on a neural network, and an emotion identification model;
a database; and
circuitry configured to:
receive, from the client terminal via the communication interface and the packet-switched network, sensor data comprising time-series data acquired by at least one sensor communicatively coupled to the client terminal, and store the sensor data in the database;
input the sensor data into an anomaly detection model comprising at least one of a recurrent neural network or a long short-term memory network to calculate an anomaly score from a multidimensional time-series tensor, and detect an abnormal value or pattern based on the anomaly score;
generate, using the data generation model, a query sentence based on the detected abnormal value or pattern, and transmit the query sentence to the client terminal via the communication interface and the packet-switched network; receive, from the client terminal via the communication interface, response data responsive to the query sentence;
generate, by inputting the response data, the sensor data, and the anomaly score into the data generation model, inference data comprising at least one of text data, voice data, or image data; and
transmit the inference data to the client terminal via the communication interface and the packet-switched network, the inference data causing the client terminal to present the inference data to the user.

2. The system according to claim 1, wherein the sensor data comprises health data acquired by a photoplethysmography sensor or a three-axis acceleration sensor built into a wearable device, the health data comprising at least one of a heart rate, a blood pressure, a body temperature, or a step count.

3. The system according to claim 1, wherein the circuitry is further configured to preprocess the sensor data by performing noise removal filtering and normalization on the time-series data, and to input the preprocessed sensor data as a tensor having dimensions of batch size, number of time steps, and number of feature dimensions into the anomaly detection model.

4. The system according to claim 1, wherein the anomaly score comprises a probability value in a range of 0.0 to 1.0 indicating the degree of deviation from the normal biological rhythm, and wherein the circuitry is configured to generate a trigger signal when the anomaly score exceeds a predetermined threshold.

5. The system according to claim 1, wherein the circuitry is further configured to estimate an emotion of the user by applying the emotion identification model to at least one of voice data, a face image, or text input received from the client terminal, and to adjust an acquisition timing of the sensor data based on the estimated emotion, such that when the estimated emotion indicates stress, the circuitry increases an acquisition frequency of the sensor data, and when the estimated emotion indicates relaxation, the circuitry decreases the acquisition frequency.

6. The system according to claim 1, wherein the circuitry is further configured to estimate an emotion of the user by applying the emotion identification model to at least one of voice data, a face image, or text input received from the client terminal, and to adjust a representation method of the inference data based on the estimated emotion, such that when the estimated emotion indicates stress, the circuitry generates the inference data in a simplified expression style, and when the estimated emotion indicates relaxation, the circuitry generates the inference data in a detailed expression style.

7. The system according to claim 1, wherein the circuitry is further configured to analyze past sensor data stored in the database associated with the user to select an optimal acquisition method for the sensor data, the selection being performed using at least one of a reinforcement learning agent or a time-series analysis algorithm.

8. The system according to claim 1, wherein the circuitry is further configured to execute a human activity recognition model taking time-series data from the at least one sensor as input to infer a current activity class of the user, and to apply a data acquisition filter according to the inferred activity class such that sensor types activated for data acquisition differ based on the activity class.

9. The system according to claim 1, wherein the circuitry is further configured to estimate an emotion of the user by applying the emotion identification model to at least one of voice data, a face image, or text input received from the client terminal, and to determine a priority of the sensor data to be acquired based on the estimated emotion.

10. The system according to claim 1, wherein the circuitry is further configured to receive geographic location information of the user from the client terminal via the communication interface, and to preferentially acquire sensor data associated with a geographic region corresponding to the geographic location information.

11. The system according to claim 1, wherein the circuitry is further configured to receive social media activity data of the user from the client terminal via the communication interface, analyze the social media activity data using a sentiment analysis model to extract a sentiment polarity and keywords, and adjust a type of the sensor data to be acquired based on the extracted sentiment polarity and keywords.

12. The system according to claim 1, wherein the circuitry is further configured to adjust a level of detail of the anomaly detection based on an importance of the sensor data, such that for sensor data having a high importance, the circuitry applies a multi-stage analysis flow using a large-scale deep neural network, and for sensor data having a low importance, the circuitry applies a simplified analysis comprising threshold determination.

13. The system according to claim 1, wherein the circuitry is further configured to apply different analysis algorithms according to a category of the sensor data.

14. The system according to claim 1, wherein the circuitry is further configured to estimate an emotion of the user by applying the emotion identification model to at least one of voice data, a face image, or text input received from the client terminal, and to adjust a length of the inference data based on the estimated emotion, such that when the estimated emotion indicates urgency, the circuitry generates a concise inference result, and when the estimated emotion indicates relaxation, the circuitry generates a detailed inference result.

15. The system according to claim 1, wherein the circuitry is further configured to determine a priority of generating the inference data based on an acquisition time associated with the sensor data, such that sensor data having a more recent acquisition time is analyzed with a higher priority.

16. The system according to claim 1, wherein the circuitry is further configured to refer to a past history of response data and corresponding inference data associated with the user stored in the database, and to select a format of the inference data based on the past history.

17. The system according to claim 1, wherein the circuitry is further configured to estimate an emotion of the user by applying the emotion identification model to at least one of voice data, a face image, or text input received from the client terminal, and to adjust an expression style of the query sentence based on the estimated emotion, such that when the estimated emotion indicates stress, the query sentence is generated in a gentle expression style, and when the estimated emotion indicates urgency, the query sentence is generated in a concise expression style.

18. A system comprising:
a communication interface configured to communicate, via a packet-switched network conforming to at least one of a 5G, Wi-Fi, or Bluetooth communication standard, with a client terminal comprising a microphone, a speaker, a camera having a CMOS image sensor, and a display;
a processor;
a random-access memory;
a memory storing a data generation model obtained by deep learning on a neural network, and an emotion identification model;
a database; and
circuitry configured to:
receive, from the client terminal via the communication interface and the packet-switched network, sensor data comprising time-series data acquired by at least one sensor communicatively coupled to the client terminal, and store the sensor data in the database;
preprocess the sensor data by performing noise removal and normalization to generate a multidimensional time-series tensor;
input the multidimensional time-series tensor into an anomaly detection model comprising at least one of a recurrent neural network or a long short-term memory network to calculate an anomaly score, and detect an abnormal value or pattern based on the anomaly score;
estimate an emotion of the user by applying the emotion identification model to at least one of voice data captured by the microphone or image data captured by the camera of the client terminal, received via the communication interface;
generate, using the data generation model, a query sentence based on the detected abnormal value or pattern, the query sentence being adapted based on the estimated emotion, and transmit the query sentence to the client terminal via the communication interface;
receive, from the client terminal via the communication interface, response data responsive to the query sentence;
generate, by inputting the response data, the sensor data, and the anomaly score into the data generation model, inference data comprising at least one of text data, voice data, or image data; and
transmit the inference data to the client terminal via the communication interface and the packet-switched network, the inference data causing the client terminal to present the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a data processing system comprising a communication interface, a memory storing a data generation model obtained by deep learning on a neural network and an emotion identification model, and a database, the method comprising:
receiving, from a client terminal via the communication interface and a packet-switched network, sensor data comprising time-series data acquired by at least one sensor communicatively coupled to the client terminal, and storing the sensor data in the database;
inputting the sensor data into an anomaly detection model comprising at least one of a recurrent neural network or a long short-term memory network to calculate an anomaly score from a multidimensional time-series tensor, and detecting an abnormal value or pattern based on the anomaly score;
generating, using the data generation model, a query sentence based on the detected abnormal value or pattern, and transmitting the query sentence to the client terminal via the communication interface and the packet-switched network;
receiving, from the client terminal via the communication interface, response data responsive to the query sentence;
generating, by inputting the response data, the sensor data, and the anomaly score into the data generation model, inference data comprising at least one of text data, voice data, or image data; and
transmitting the inference data to the client terminal via the communication interface and the packet-switched network, the inference data causing the client terminal to present the inference data to the user.
